(19) United States
(12) Patent Application Publication
(10) Pub. No.: US 2020/0095540 A1
(43) Pub. Date: Mar. 26, 2020

(54) METHODS OF PRODUCING THREE-DIMENSIONAL CELL CULTURE MICROENVIRONMENTS

(71) Applicant: BIOACTIVE REGENERATIVE THERAPEUTICS, INC., Two Harbors, MN (US)

(72) Inventors: John Harold GUBBE, (US); Yi Wen CHAI, Minneapolis, MN (US); Mary Beth Klausen, Two Harbors, MN (US)

(73) Assignee: BIOACTIVE REGENERATIVE THERAPEUTICS, INC., Two Harbors, MN (US)

(21) Appl. No.: 16/582,383

(22) Filed: Sep. 25, 2019

Related U.S. Application Data
(60) Provisional application No. 62/736,067, filed on Sep. 25, 2018.

(51) Int. Cl. C12N 5/00; C12M 1/12

(52) U.S. Cl. CPC C12N 5/0062; C12N 5/0068; C12N 2533/80; C12N 2533/30; C12N 2539/00; C12M 25/14

(57) ABSTRACT

Disclosed herein are methods of producing a three-dimensional cell culture microenvironment, which may include exposing a lyophilized polycation coupon to a polyanion and a fluid medium. The lyophilized polycation coupon may optionally include a void volume, and the polyanion may optionally be contained within a slurry. Such methods may also include the steps of evaporating off the fluid medium of the slurry to produce a dried three-dimensional architecture, administering a hydrating fluid to the dried three-dimensional architecture, and producing the three-dimensional cell culture microenvironment. Also disclosed herein are three-dimensional cell culture microenvironments formed by various processes, as well as kits which include such three-dimensional cell culture microenvironments provided in a multi-well plate, and a hydrating fluid.

### CROSS-REFERENCE TO RELATED APPLICATIONS

This application claims priority to and benefit of U.S. Provisional Application Ser. No. 62/736,067, filed Sep. 25, 2018, entitled “Methods of Producing Three-Dimensional Cell Culture Microenvironments,” which is incorporated herein by reference in its entirety.

### SUMMARY

The fields of regenerative medicine and tissue engineering focus on repairing and regenerating tissue lost or damaged due to injury, disease, or congenital anomalies. Effective regenerative medicine constructs can be created when knowledge of healthy tissue composition, organization (at cellular and molecular levels), and complex biologic functions are joined with knowledge of biomimetic materials and integrated with therapeutic advantages provided by various cell phenotypes and soluble signaling factors. Chemical, structural, mechanical, and biologic properties of such materials can be controlled to provide customized, biomimetic microenvironments within which populations of pluripotent cells can expand and differentiate to therapeutically useful phenotypes. Furthermore, the microenvironments in which these cells expand and differentiate can be formed by various methods and with various processes.

Several methods have previously been used to form three-dimensional cell culture microenvironments. However, many of these processes and methods have included multiple difficult or laborious steps, and error in any step can result in waste or a prolonged process. In addition, many previous processes and methods have produced three-dimensional cell culture microenvironments that are difficult to fit into small cell culture plates or other vehicles without additional, laborious steps. Furthermore, larger three-dimensional cell culture microenvironments may not be translational, because they may not be easily injectable in vivo. Thus, there is a need for improved methods and processes for producing three-dimensional cell culture microenvironments.

Disclosed herein are methods of producing a three-dimensional cell culture microenvironment, which can be used for in vitro and in vivo cultures of cells or, optionally, other therapeutics, such as drugs, that are acellular. Such methods are adaptable to provide customized microenvironments within which pluripotent and/or therapeutic cell types, or other therapeutics, may be embedded. Three-dimensional cell culture microenvironments produced by such methods and processes, as well as kits containing these microenvironments, are also disclosed.

In some embodiments, a method of producing a three-dimensional cell culture microenvironment may include exposing a lyophilized polycation coupon to a polyanion and a fluid medium. The lyophilized polycation coupon may optionally include a void volume, and the polyanion may optionally be contained within a slurry. The method may further include the steps of evaporating off the fluid medium to produce a dried three-dimensional architecture, administering a hydrating fluid to the dried three-dimensional architecture, and producing the three-dimensional cell culture microenvironment. In embodiments wherein the polyanion is contained within a slurry, the method may further include the step of evaporating off the fluid medium of the slurry to produce the dried three-dimensional architecture.

In certain embodiments, a three-dimensional cell culture microenvironment may be formed by a particular process. The process may include, among other things, exposing a lyophilized polycation coupon to a polyanion and a fluid medium. The lyophilized polycation coupon may optionally include a void volume, and the polyanion may optionally be contained within a slurry. The process may further include the steps of evaporating off the fluid medium to produce a dried three-dimensional architecture, administering a hydrating fluid to the dried three-dimensional architecture, and producing the three-dimensional cell culture microenvironment.

In some embodiments, a kit may include a dried three-dimensional architecture provided in a multi-well plate or other vehicle, and a hydrating fluid. In certain embodiments, the dried three-dimensional architecture of the kit may be produced by the methods and processes described herein.

### BRIEF DESCRIPTION OF THE FIGURES

FIG. 1 is a schematic illustration of a three-dimensional cell culture microenvironment, in accordance with the present disclosure.

FIG. 2A is a schematic illustration of three embodiments of a three-dimensional cell culture microenvironment, the embodiments having void volumes with inverted-cone shapes of various volumes and depths, in accordance with the present disclosure.

FIG. 2B is a schematic illustration of five embodiments of a three-dimensional cell culture microenvironment, the embodiments having void volumes with substantially cylindrical shapes of various volumes and depths, in accordance with the present disclosure.

FIG. 2C is a schematic illustration of six embodiments of a three-dimensional cell culture microenvironment, the embodiments having void volumes with substantially elliptical shapes of various volumes and depths, in accordance with the present disclosure.

### DETAILED DESCRIPTION

Disclosed herein are methods and processes for producing a three-dimensional cell culture microenvironment. Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention, which will be limited only by the appended claims. Unless otherwise defined, all technical and scientific terms used herein have the same meanings as commonly understood by one of ordinary skill in the art. Although any methods and materials similar or equivalent to those described herein may be used in the practice or testing of embodiments of the present invention, the preferred methods, devices, and materials are now described. All publications mentioned herein are incorporated by reference in their entireties. Nothing herein is to be construed as an admission that the invention is not entitled to antedate such disclosure by virtue of prior invention.

It must be noted that as used herein, and in the appended claims, the singular forms “a,” “an,” and “the” include plural references unless the context clearly dictates otherwise. Thus, for example, a reference to a “polycation” is a reference to one or more polycations and equivalents thereof known to those skilled in the art. Unless defined otherwise, all technical and scientific terms used herein have the same meanings as commonly understood by one of ordinary skill in the art. As used herein, the term “comprising” means “including, but not limited to.”

As used herein, all claimed numeric terms are to be read as being preceded by the term, “about,” which means plus or minus 10% of the numerical value of the number with which it is being used. Therefore, a claim to “50 mm” means “about 50 mm” and encompasses the range of 45 mm-55 mm.

As used herein, the term “consists of” or “consisting of” means that the device or method includes only the elements, steps, or ingredients specifically recited in the particular claimed embodiment or claim.

In embodiments or claims where the term comprising is used as the transition phrase, such embodiments can also be envisioned with replacement of the term “comprising” with the terms “consisting of” or “consisting essentially of.”

The term “macromolecule” as used herein includes, but is not limited to, any molecule of large molecular weight or formed by polymerization of smaller molecules, such as polymeric monomers/dimers, etc., proteins, lipids, polysaccharides or nucleic acids.

The term “solvating fluid” as used herein includes, but is not limited to, any fluid that dissolves a solute or solutes. Solvating fluid may also be referred to as a fluid of hydration, or a “hydrating fluid.”

The term “biocompatible” as used herein refers to a composition being harmonious with life; not having toxic or injurious effects on biological function at molecular, cellular or tissue levels; and compatible with living tissue. While parameters of biocompatibility may be measured by various means, compositions that do not elicit an immune response (or only elicit a minimal response) are biocompatible. Similarly, compositions that are not toxic to an organism or to juxtaposed cells are biocompatible.

The term “composition” includes those which are generated by polycationic and polyanionic macromolecules. The compositions may include additional components and/or a volume of fluid.

The term “anhydrous” refers to a composition, macromolecule, molecule, particle, material, or substance having no water. The term “anhydrous” is used interchangeably herein with “dry.”

The term “plurality” encompasses multiple species. As used herein, for example, the term “plurality” may encompass multiple species of polyanionic macromolecules, multiple species of polycationic macromolecules, multiple molecules of a homogeneous species of polyanionic macromolecules, and multiple molecules of a homogeneous species of polycationic macromolecules.

The term “exposing” as used herein is interchangeable with related terms such as “partially exposing,” “partially soaking,” “substantially soaking,” “partially saturating,” and “substantially saturating.” These terms refer to the exposure of at least a portion of one component to at least a portion of a second component. Full exposure may be encompassed by the term, but is not required.

The term “coupon” as used herein refers to a three-dimensional structure or construct of dried materials. A “coupon” may also be described as a scaffold or matrix. “Coupon” and its synonyms are used to describe a small three-dimensional piece of dried material. In one non-limiting example, a coupon is a dry three-dimensional scaffold that fits into a single well of a multi-well plate, or into a vehicle (e.g., a delivery vehicle such as a syringe). Without wishing to be bound by theory, the coupon may function as an artificial extracellular matrix for cultured cells or for other therapeutics, such as drugs or other compounds that may be acellular.

In some embodiments, a composition may comprise one or more polycations and/or one or more polyanions. In each embodiment described herein, the use of a polycation and a polyanion in the methods and processes may be interchangeable. For example, in one embodiment, a lyophilized polycation coupon may be exposed to a polyanion, while in another embodiment, a lyophilized polyanion coupon may be exposed to a polycation. The terms are distinct, but in any step employing a polyanion, a polycation may alternatively be used. In some embodiments, the one or more polycations and/or one or more polyanions are anhydrous. Examples of polyanions include, but are not limited to, dextran sulfate and glycosaminoglycans, such as dermatan sulfate, keratan sulfate, heparan sulfate, and hyaluronan, or a combination thereof. For example, the polyanions may include dextran sulfate and an additional glycosaminoglycan, such as hyaluronan. In some embodiments, the polyanion(s) may have a Feret diameter of less than about 1,000 μm. Examples of polycations may include, but are not limited to, cellulose, chitosan, any other linear polysaccharide capable of being protonated, or a combination thereof. For example, the polycations may include chitosan and cellulose.

### Methods and Processes for Producing a Three-Dimensional Cell Culture Microenvironment

The methods and processes disclosed herein may be used to create three-dimensional microenvironments. These three-dimensional microenvironments may be useful for cell culture, but they may also be useful for holding and/or delivering acellular therapeutics (such as drugs, in one non-limiting example). These three-dimensional microenvironments are described herein as cell culture microenvironments, but as one skilled in the art would appreciate, they may have utility outside of cell culture. Accordingly, where the term “cell culture” is used herein, it is understood that the term may or may not apply in every embodiment—that is, that a three-dimensional microenvironment suitable for cell culture may also, or alternatively, be suitable for use with acellular compounds. In other words, the description of a “three-dimensional cell culture microenvironment” is not intended to limit the microenvironment to use in cell culture.

Without wishing to be bound by theory, the embodiments disclosed herein may provide a three-dimensional cell culture microenvironment suitable for use in high-content analysis systems. Particularly, the embodiments disclosed herein may provide the ability to image the microenvironment from the bottom of a cell culture plate, and the ability to incorporate the microenvironment into a 96-well plate or smaller. Such microenvironments may be useful for disease modeling and regenerative medicine research, for example. Furthermore, the embodiments disclosed herein may be suitable for use with a relatively low number of cells (lower limit about 12,000) embedded therein. Moreover, the embodiments disclosed herein may provide simplicity and ease of use, with the kits disclosed herein able to be set up in as little as about five minutes.

In some embodiments, a method of producing a three-dimensional cell culture microenvironment may include exposing a lyophilized polycation coupon to a polyanion and a fluid medium. In some embodiments, the lyophilized polycation coupon may optionally include a void volume, and the polyanion may optionally be contained within a slurry. In certain embodiments, the void volume may be a central or centralized void volume, while in other embodiments, the void volume may be substantially central or centralized, or it may be off-center. In some embodiments, the lyophilized polycation coupon may include (i.e., have incorporated into one or more of its components) one or more biologically active agents, as described herein.

In certain embodiments, the lyophilized polycation coupon may be formed by freezing a solution comprising a polycation and a surfactant, followed by lyophilizing the solution. In some embodiments, the step of freezing may comprise flash-freezing and/or global freezing. The flash-freezing and/or global freezing may occur, for example, using a freezer at about −25° C., or using liquid nitrogen. In some embodiments, the surfactant may include, for example, poloxamers (pluronic f68, for example), polysorbates, alkylphenols, organosulfates, or any combination thereof. In certain embodiments, the solution may be selected from the group consisting of water, saline, any solution suitable for human injection, or a combination thereof. In some embodiments, the step of lyophilizing the solution may be performed using a vacuum. In an embodiment, the fluid medium may be selected from the group consisting of alcohol, ethanol, methanol, isopropanol, or a combination thereof.

In certain embodiments, the lyophilized polycation coupon may have a plurality of internal void spaces. The plurality of internal void spaces may be defined by a network of thin partitions. In some embodiments, the thin partitions may comprise incomplete partitions. In an embodiment, the internal void space(s) of a single lyophilized coupon may have a total volume of from about 10 mm³ to about 600 mm³. The internal void space may have a volume of, for example, about 10 mm³, about 20 mm³, about 30 mm³, about 40 mm³, about 50 mm³, about 60 mm³, about 70 mm³, about 80 mm³, about 90 mm³about 100 mm³, about 110 mm³, about 120 mm³, about 130 mm³, about 140 mm³, about 150 mm³, about 160 mm³, about 170 mm³, about 180 mm³, about 190 mm³, about 200 mm³, about 210 mm³, about 220 mm³, about 230 mm³, about 240 mm³, about 250 mm³, about 260 mm³, about 270 mm³, about 280 mm³, about 290 mm³, about 300 mm³, about 310 mm³, about 320 mm³, about 330 mm³, about 340 mm³, about 350 mm³, about 360 mm³, about 370 mm³, about 380 mm³, about 390 mm³, about 400 mm³, about 410 mm³, about 420 mm³, about 430 mm³, about 440 mm³, about 450 mm³, about 460 mm³, about 470 mm³, about 480 mm³, about 490 mm³, about 500 mm³, about 510 mm³, about 520 mm³, about 530 mm³, about 540 mm³, about 550 mm³, about 560 mm³, about 570 mm³, about 580 mm³, about 590 mm³, about 600 mm³, or any range between any two of these values, including endpoints.

In some embodiments, the lyophilized polycation coupon may have a cylindrical shape having a diameter and a height. The lyophilized polycation coupon may also optionally contain a void volume, resulting in the three-dimensional cell culture microenvironment having the void volume. In certain embodiments, the void volume may be a central or centralized void volume, while in other embodiments, the void volume may be substantially central or centralized, or it may be off-center. In an embodiment, the void volume is a central void volume. In certain embodiments, the void volume may have an inverted-cone shape, while in other embodiments the void volume may have a substantially cylindrical shape, a substantially rectangular shape, a substantially elliptical shape, or any other shape known in the art. In an embodiment, the void volume may be defined by a diameter and an apex. The apex may, for example, terminate at from about 0.5 mm to about 4 mm from a bottom surface of the cylindrical shape of the lyophilized polycation coupon. In some embodiments, the apex may terminate at, for example, about 0.5 mm from a bottom surface of the cylindrical shape of the lyophilized polycation coupon, about 1 mm from a bottom surface of the cylindrical shape of the lyophilized polycation coupon, about 1.5 mm from a bottom surface of the cylindrical shape of the lyophilized polycation coupon, about 2 mm from a bottom surface of the cylindrical shape of the lyophilized polycation coupon, about 2.5 mm from a bottom surface of the cylindrical shape of the lyophilized polycation coupon, about 3 mm from a bottom surface of the cylindrical shape of the lyophilized polycation coupon, about 3.5 mm from a bottom surface of the cylindrical shape of the lyophilized polycation coupon, about 4 mm from a bottom surface of the cylindrical shape of the lyophilized polycation coupon, or any range between any two of these values, including endpoints.

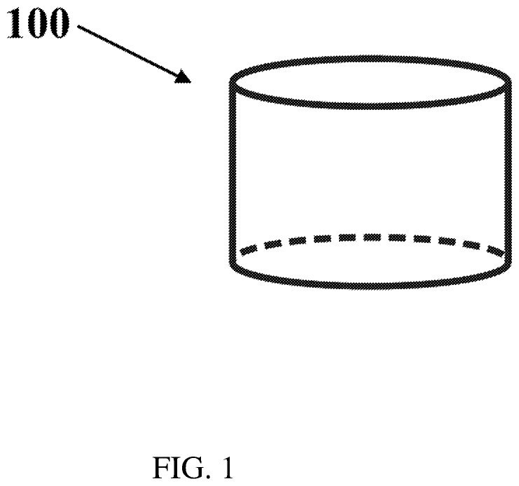

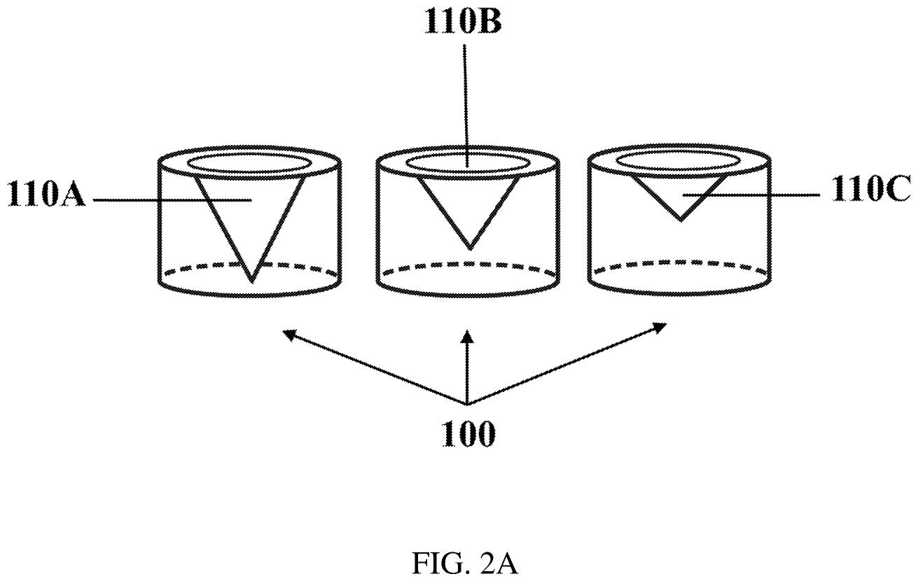

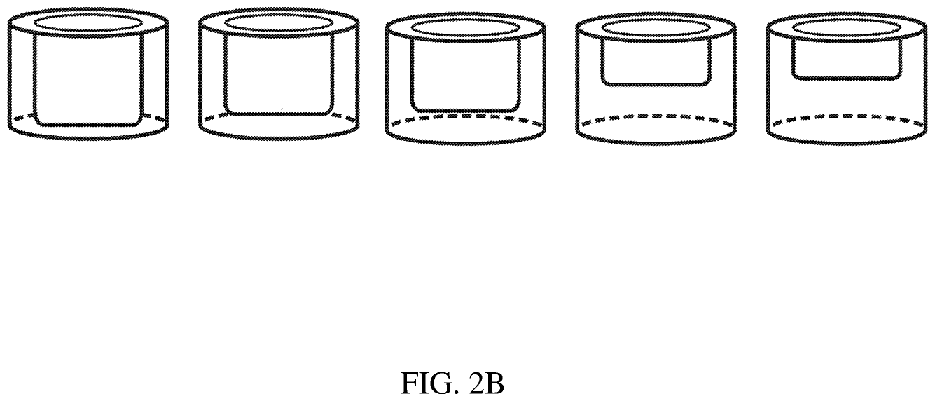

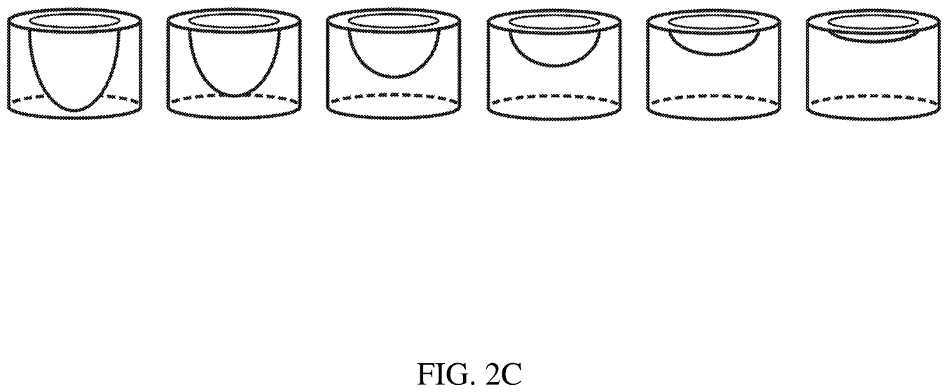

FIG. 1 is a schematic illustration of a three-dimensional cell culture microenvironment 100 formed by the methods and processes described herein. FIG. 2A is a schematic illustration of three embodiments of a three-dimensional cell culture microenvironment 100, the three-dimensional cell culture microenvironments 100 having void volumes 110A, 110B, and 110C with inverted-cone shapes of various volumes and depths (decreasing in volume and depth from the left-most inverted-cone shaped void volume 110A, through the middle inverted cone-shaped void volume 110B, to the right-most inverted cone-shaped void volume 110C). Similarly, FIG. 2B is a schematic illustration of five embodiments of a three-dimensional cell culture microenvironment, the three-dimensional cell culture microenvironments having void volumes with substantially cylindrical shapes of various volumes and depths (decreasing in volume and depth from left to right). Finally, FIG. 2C is a schematic illustration of six embodiments of a three-dimensional cell culture microenvironment, the three-dimensional cell culture microenvironments having void volumes with substantially elliptical shapes of various volumes and depths (decreasing in volume and depth from left to right).

In some embodiments, the lyophilized polycation coupon may have a diameter from about 5 mm to about 12 mm. The diameter of the lyophilized polycation coupon may be, for example, about 5 mm, about 5.2 mm, about 5.4 mm, about 5.6 mm, about 5.8 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, or any range between any two of these values, including endpoints. In certain embodiments, the lyophilized polycation coupon may have a height from about 0.5 mm to about 15 mm. The height of the lyophilized polycation coupon may be, for example, about 0.5 mm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, about 15 mm, or any range between any two of these values, including endpoints.

In certain embodiments, the lyophilized polycation coupon may include one or more biologically active agents. The biologically active agents may include, for example, serum albumin, peptide fragments, autologous and/or non-autologous serum and serum components, fetal bovine serum, albumin, growth factors, morphogens, hormones, cytokines, vitamins, amino acids, glycerol phosphate, normal saline, autologous interstitial fluid, extracellular matrix glycoproteins, proteoglycans, glycosaminoglycans, cytotoxic agents, therapeutic pharmaceutical compounds, exendin-4, betacellulin, peptides that bind an α5β1 integrin, islet neogenesis-associated protein fractions, islet neogenesis-associated proteins, collagen, laminate, fibronectin, or combinations thereof. In an embodiment, the lyophilized polycation includes one or more growth factors.

In certain embodiments, the method of producing a three-dimensional cell culture microenvironment may further include evaporating off the fluid medium to produce a dried three-dimensional architecture. In some embodiments, the step of evaporating may be performed using a vacuum. In an embodiment, the vacuum may be used at about 30° C.

In some embodiments, the method of producing a three-dimensional cell culture microenvironment may further include administering a hydrating fluid to the dried three-dimensional architecture. In certain embodiments, the hydrating fluid may comprise one or more components selected from a polyanion, a polycation, hyaluronan, ionizing solvents, water, culture medium, LMD dextran 40 in dextrose, sodium chloride injection solution, glycerol phosphate disodium salt, or any combination thereof.

In some embodiments, the hydrating fluid may optionally comprise one or more types of living cells. The living cells may include, for example, pluripotent cells, islet cells, adrenal cells, mesenchymal stem cells, thyroid cells, parathyroid cells, parafollicular cells, pinealocytes, pituitary cells, neurosecretory cells, endocrine progenitor cells, induced pluripotent stem cells, or a combination thereof. In certain embodiments, the hydrating fluid may comprise one or more biologically active agents. The biologically active agents may include, for example, serum albumin, peptide fragments, autologous and/or non-autologous serum and serum components, fetal bovine serum, albumin, growth factors, morphogens, hormones, cytokines, vitamins, amino acids, glycerol phosphate, normal saline, autologous interstitial fluid, extracellular matrix glycoproteins, proteoglycans, glycosaminoglycans, cytotoxic agents, therapeutic pharmaceutical compounds, exendin-4, betacellulin, peptides that bind an α5β1 integrin, islet neogenesis-associated protein fractions, islet neogenesis-associated proteins, collagen, laminate, fibronectin, or combinations thereof. In certain embodiments, the step of administering the hydrating fluid may be performed under sterile conditions. In one embodiment, the step of administering the hydrating fluid may be performed by drop loading the hydrating fluid into the void volume.

In an embodiment, the hydrating fluid may optionally comprise one or more acellular therapeutics. These acellular therapeutics may optionally be combined with the one or more types of living cells described herein. The acellular therapeutic(s) may include, for example, cytotoxic agents, pharmaceutical compounds, or a combination thereof.

In certain embodiments, the method may further include producing the three-dimensional cell culture microenvironment. In an embodiment, the three-dimensional cell culture microenvironment may be a hydrogel complexed with insoluble polyelectrolytic fibers.

In certain embodiments, a three-dimensional cell culture microenvironment as described herein may be formed by a particular process. The process may include any of the steps or have any of the characteristics of the methods described herein. The process may include, among other things, exposing a lyophilized polycation coupon to a polyanion and a fluid medium, as described herein. In certain embodiments in which the polyanion and the fluid medium comprise a slurry, the process may further include the steps of evaporating off the fluid medium to produce a dried three-dimensional architecture, administering a hydrating fluid to the dried three-dimensional architecture, and producing the three-dimensional cell culture microenvironment, as described herein.

### Kits Including Three-Dimensional Cell Culture Microenvironments

In some embodiments, a kit may include a dried three-dimensional architecture provided in a vehicle such as a multi-well plate, and a hydrating fluid. In certain embodiments, the dried three-dimensional architecture of the kit may be produced by the methods and processes described herein. For example, the dried three-dimensional architecture may be produced by a method comprising exposing a lyophilized polycation coupon to a polyanion, and a fluid medium, and evaporating the fluid medium. The lyophilized polycation coupon may be formed by the methods described herein, including the steps of freezing a solution having a polycation and a surfactant, and lyophilizing the solution.

In certain embodiments, the vehicle included in the kit may comprise a multi-well plate. The multi-well plate may be any type of multi-well plate known in the art. The multi-well plate may include, for example, a 4-well plate, a 6-well plate, an 8-well plate, a 12-well plate, a 24-well plate, a 48-well plate,a 96-well plate, a 384-well plate, a 1,536-well plate, or any combination thereof.

In certain embodiments, the vehicle included in the kit may comprise a delivery vehicle such as a syringe or a vial. The delivery vehicle may have a total capacity of, for example, 1 cc, 2 cc, 3 cc, 4 cc, 5 cc, 6 cc, 7 cc, 8 cc, 9 cc, 10 cc, or any range between any two of these values, including endpoints.

In some embodiments, the kit may further include instructions for use. In an embodiment, the instructions for use may include descriptions of the methods and/or processes by which the kit's components have been created, including the methods and/or processes disclosed herein. The instructions for use may further include instructions for applying the hydrating fluid to the dried three-dimensional architecture. In certain embodiments, the instructions for use may include statements that the dried three-dimensional architecture has been produced by a method comprising exposing a lyophilized polycation coupon to a polyanion, and a fluid medium, and evaporating the fluid medium. Similarly, the instructions for use may include statements that the lyophilized polycation coupon has been formed by the methods described herein, including the steps of freezing a solution having a polycation and a surfactant, and lyophilizing the solution.

Because the invention disclosed herein may be embodied in other specific forms without departing from the spirit or general characteristics thereof, some of which have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

1.-69. (canceled)

70. A method of producing a three-dimensional cell culture microenvironment, the method comprising:
exposing a lyophilized polycation coupon to a polyanion and a fluid medium;
evaporating off the fluid medium to produce a dried three-dimensional architecture;
administering a hydrating fluid to the dried three-dimensional architecture; and
producing the three-dimensional cell culture microenvironment.

71. The method of claim 70, wherein the lyophilized polycation coupon comprises a void volume.

72. The method of claim 70, wherein the lyophilized polycation coupon is formed by freezing a solution having a polycation and a surfactant, followed by lyophilizing the solution.

73. The method of claim 72, wherein the surfactant is selected from the group consisting of a poloxamer, a polysorbate, an alkylphenol, an organosulfate, pluronic f68, and combinations thereof.

74. The method of claim 70, wherein the lyophilized polycation coupon has a plurality of internal void spaces defined by a network of thin, incomplete partitions, and wherein the total volume of the plurality of internal void spaces is from about 40 mm³ to about 600 mm³.

75. The method of claim 71, wherein the lyophilized polycation coupon has a cylindrical shape having a diameter and a height; and wherein the void volume of the lyophilized polycation coupon is defined by a diameter and an apex, the apex terminating from about 0.5 mm to about 4 mm from a bottom surface of the cylindrical shape of the lyophilized polycation coupon.

76. The method of claim 75, wherein the diameter of the lyophilized polycation coupon is from about 5 mm to about 12 mm, and the height of the lyophilized polycation is from about from about 0.5 mm to about 15 mm.

77. The method of claim 72, wherein the polycation is selected from the group consisting of chitosan, cellulose, any other linear polysaccharide capable of being protonated, and any combination thereof, and wherein the polyanion is selected from the group consisting of hyaluronan, dextran sulfate, glycosaminoglycans, dermatan sulfate, keratan sulfate, heparan sulfate, and combinations thereof.

78. The method of claim 72, wherein the lyophilizing is performed using a vacuum, and wherein the evaporating is performed using a vacuum at about 30° C.

79. The method of claim 70, wherein the fluid medium is selected from the group consisting of alcohol, ethanol, methanol, isopropanol, and combinations thereof.

80. The method of claim 70, wherein the hydrating fluid comprises one or more components selected from the group consisting of a polyanion, a polycation, hyaluronan, ionizing solvents, water, culture medium, LMD dextran 40 in dextrose, sodium chloride injection solution, glycerol phosphate disodium salt, and combinations thereof.

81. The method of claim 70, wherein the hydrating fluid comprises living cells selected from the group consisting of pluripotent cells, islet cells, adrenal cells, mesenchymal stem cells, thyroid cells, parathyroid cells, parafollicular cells, pinealocytes, pituitary cells, neurosecretory cells, endocrine progenitor cells, induced pluripotent stem cells, and combinations thereof.

82. The method of claim 70, wherein the hydrating fluid comprises one or more biologically active agents selected from the group consisting of serum albumin, peptide fragments, autologous and non-autologous serum and serum components, fetal bovine serum, albumin, growth factors, morphogens, hormones, cytokines, vitamins, amino acids, glycerol phosphate, normal saline, autologous interstitial fluid, extracellular matrix glycoproteins, proteoglycans, glycosaminoglycans, cytotoxic agents, therapeutic pharmaceutical compounds, exendin-4, betacellulin, peptides that bind an α5β1 integrin, islet neogenesis-associated protein fractions, islet neogenesis-associated protein, collagen, laminate, fibronectin, and combinations thereof.

83. The method of claim 70, wherein the three-dimensional cell culture microenvironment is a hydrogel complexed with insoluble polyelectrolytic fibers.

84. A three-dimensional cell culture microenvironment formed by the process of:
exposing a lyophilized polycation coupon to a polyanion and a fluid medium;
evaporating off the fluid medium to produce a dried three-dimensional architecture;
administering a hydrating fluid to the dried three-dimensional architecture; and
producing the three-dimensional cell culture microenvironment.

85. The three-dimensional cell culture microenvironment of claim 84, wherein the lyophilized polycation coupon comprises a void volume.

86. A kit comprising:
a dried three-dimensional architecture provided in a multi-well plate;
a hydrating fluid; and
instructions for use.

87. The kit of claim 86, wherein the dried three-dimensional architecture is produced by a method comprising:
exposing a lyophilized polycation coupon to a polyanion and a fluid medium; and
evaporating off the fluid medium.

88. The kit of claim 87, wherein the lyophilized polycation coupon comprises a void volume.

89. The kit of claim 87, wherein the lyophilized polycation coupon is formed by a method comprising:
freezing a solution having a polycation and a surfactant; and
lyophilizing the solution.
